(19) United States
(12) Patent Application Publication
(10) Pub. No.: US 2020/0113726 A1
(43) Pub. Date: Apr. 16, 2020

(54) Urine Collector and Kit Comprising the Collector

(71) Applicant: Kang Chien Medical Co., Ltd., Taipei City (TW)

(72) Inventors: Ming-Chi Yuan, Taipei City (TW); Te-Chou Yang, Kaohsiung City (TW)

(21) Appl. No.: 16/595,021

(22) Filed: Oct. 7, 2019

(30) Foreign Application Priority Data
Oct. 16, 2018 (TW) 107136347

(51) Int. Cl. A61F 5/453; A61F 5/44

(52) U.S. Cl. CPC A61F 5/453; A61F 5/4404

(57) ABSTRACT

A urine kit includes a collector and a piece of underwear. The collector includes a container and a short guide tube. The container defines a temporary storage space therein and an opening communicating with the temporary storage space, and is provided with an outlet tube communicating with the temporary storage space. The short guide tube, which is in watertight engagement with a portion of the container around the opening, includes a connection section having a neck portion and a grooved portion. The short guide tube has an inner surface, to which the gradient of a tangent thereof changes as being made along the inner the surface, such that an annular surface area with a minimum diameter is formed at the neck portion; an inflexion surface area is formed at the grooved portion; a flow directing surface area extends from the inflexion surface area.

### FIELD OF THE INVENTION

The present invention relates to a urine kit, a collector, and a short guide tube used in the collector, and more particularly, to a urine kit and a collector that can be used easily.

### BACKGROUND OF THE INVENTION

For old people or bedridden patients, or persons who drive their cars on a highway, there is a need to have a urine collector. However, conventional urine collectors for male users have some disadvantages. For example, the collectors are difficult to clean, causing breeding of germs and producing odors. The collectors made of hard materials often bring discomfort to users. Also, in use, the collectors would compress the testicles of a male user. Besides, the urine contained in the collectors may flow back towards the genital organ of a male, bedridden user. If the collectors are unable to closely contact the skin around the male genital organ, the urine contained in the collectors may flow out to wet the body, clothes and bedding of the user.

As shown in FIG. 14, the urine collector disclosed in U.S. Pat. No. 5,478,334 includes a pouch 10 defining an inlet 11, around which a circular flange 12 is furnished, and a condom 23 with a fixture 22, which can be fitted into the pouch 10.

The condom 23 can be snugly fitted around the male genital organ (penis) of a male user, so that a watertight engagement between the condom and the male genital organ can be ensured. However, after the genital skin is tightly covered with a sheath made of an airtight and impervious material for a long time, a slight itching and redness on the skin may occur. Due to immune system response, the skin may swell or inflame, and even cause ulceration.

As shown in FIG. 15, the external urinary device disclosed in U.S. Pat. No. 4,020,843 employs a hydrophobic platform 12 to prevent the urine from flowing back, so that a tight fit between the device and a user's genital organ is not required. However, the user is not permitted to lie down while using the device. Although the platform 12 slopes slightly downwards, the device can be easily affected to change the position of the platform 12. When the platform becomes sloped upwards, the urine contained in the lower compartment can flow back to wet the body, clothes and the bedding of the user.

The urinary collector disclosed in U.S. Pat. No. 5,300,052 includes a waterproof soft bag, in which multiple hydrophobic beads forming unidirectional channels are arranged. However, the device is complicated in structure, which usually leads to a high manufacturing cost. Another disadvantage is that the device cannot be cleaned and reused, which in turn causes a large amount of waste, and thus does not meet the trend of environmental protection.

For the urinary device disclosed in China Invention Patent 102784025, due to the device requiring a tight fit, adverse effects as illustrated in the prior-art device shown in FIG. 14 would be caused.

Moreover, because of improvement in medical standards and the number of elder people being on the increase, the demand for long-term care increases. There is a need to provide a urine collector that is simple in structure and does cost a lot of money, and can be used in various conditions such as standing, sitting and lying. Also, the urine collector can avoid leakage or overflow of urine and is easy to clean and can be used without a tight fit, so that the genital skin can be prevented from damages, and the user may feel more comfortable.

### SUMMARY OF THE INVENTION

One object of the present invention is to provide a urine kit, which can be used easily by a user who is out of a room, and can be concealed easily to maintain dignity.

Another object of the present invention is to provide a urine collector that is simple in structure, so that the manufacturing cost can be reduced significantly.

A further object of the present invention is to provide a urine collector that is suitable to patients under any postural conditions; no matter which body posture is taken, overflow or leakage of urine can be avoided.

A still further object of the present invention is to provide a detachable urine collector, so that the interior of the collector can be cleaned and disinfected thoroughly to prevent breeding of germs and release of odor, and the collector can be used many times to meet the trend of environmental protection.

A yet still further object of the present invention is to provide a urine collector, which includes a short guide tube provided with a buffer section that can prevent the testicles of a male user from being compressed, so that the urine collector can be used in comfort.

A yet still further object of the present invention is to provide a short guide tube for a urine collector, which can be used in comfort without requiring a tight fit, so that skin redness, broken skin, ulceration, and decubitus can be avoided.

A yet still further object of the present invention is to provide a short guide tube for a urine collector, which can be used easily without requiring a tight fit to facilitate carers to take care of patients.

To achieve the above objects, the urine collector may comprise a container and a short guide tube. The container has a first wall and a second wall opposite to the first wall, and defines a temporary storage space between the first and second walls. The first wall faces a user's lower abdomen and defines an opening communicating with the temporary storage space. The container is provided with an outlet tube communicating with the temporary storage space and extending from the first wall and/or the second wall. The short guide tube, which in watertight engagement with a portion of the first wall around the opening, includes a connection section and a main section extending from the connection section and located in the temporary storage space. The main section is formed with a bottom downwardly curved edge that is distal from the connection section and at a level higher than the outlet tube. The main section, which functions as a flow blocking wall, can prevent the urine contained in the temporary storage space from overflow to enter the short guide tube. The urine discharged from the user can enter the temporary storage space via the bottom downwardly curved edge of the short guide tube.

The urine kit may comprise the aforementioned urine collector and a piece of underwear including a main body and an auxiliary part. The main body has a crotch section. The auxiliary part is disposed in the underwear to define a space between the crotch section of the underwear and the auxiliary part for accommodating the container and the short guide tube. A first hole is defined at the auxiliary part or the underwear to allow the short guide tube to pass therethrough. A second hole having a dimension greater than the outlet tube is defined at the auxiliary part and/or the underwear.

According to one aspect of the present invention, the container can be constructed of a distal shell and a proximal shell. The distal shell has a circumferential edge in addition to the aforementioned second surface. The proximal shell has the aforementioned first surface and a circumferential sealing portion capable of being watertight engagement with the circumferential edge of the distal shell.

According to another aspect of the invention, the short guide tube can be made of a flexible material. The short guide tube is further provided with a buffer section extending from the connection section in a direction opposite to the main section. The buffer section can be brought in elastic contact with a male user's lower abdomen such that a space is defined between the first wall and the user's lower abdomen so as to accommodate the testicles of the male user.

Compared to conventional devices, the present invention is advantageous in that the urine can be prevented from entering the short guide tube regardless of a male user's postural condition: standing, sitting or lying; particularly, the short guide tube can be used with a loose fit onto the male genital organ to ensure comfort and prevent skin allergies, inflammation or ulceration; the short guide tube can be detached from a container of the collector to facilitate cleaning, and this can make the collector be reusable; moreover, the main section of the short guide tube is configured with a curved surface extending towards the outlet tube and conforming to the male genital organ in natural state; the short guide tube includes a buffer section to prevent the testicles of a male user from being compressed, thus increasing comfort; the urine kit includes a piece of underwear to facilitate a male user using the kit on the occasion of going out and to offer the user privacy and dignity.

### BRIEF DESCRIPTION OF THE DRAWINGS

FIG. 1 shows a 3-dimensional view of a first embodiment of a urine collector of the present invention.

FIG. 2 shows a side view of the first embodiment of the urine collector.

FIG. 3 shows an exploded view of the first embodiment of the urine collector.

FIG. 4 shows a sectional view of a short guide tube taken along line 4-4 in FIG. 3.

FIG. 5 shows a working view of a urine kit, which includes a urine collector according to the first embodiment.

FIG. 6 shows an exploded view of a second embodiment of the urine collector.

FIG. 7 shows a working view of a urine kit, which includes a urine collector according to the second embodiment.

FIG. 8 shows an exploded view of a third embodiment of the urine collector.

FIG. 9 shows a schematic view of a fourth embodiment of the urine collector.

FIG. 10 shows an enlarged fragmentary view of the urine collector shown in FIG. 9.

FIG. 11 shows a working view of a urine kit, which includes a urine collector according to the fourth embodiment.

FIG. 12 shows a schematic view of an adaptive member used in the fourth embodiment of the urine collector.

FIG. 13 shows a schematic view of a fifth embodiment of the urine collector.

FIG. 14 shows a schematic view of a prior-art device.

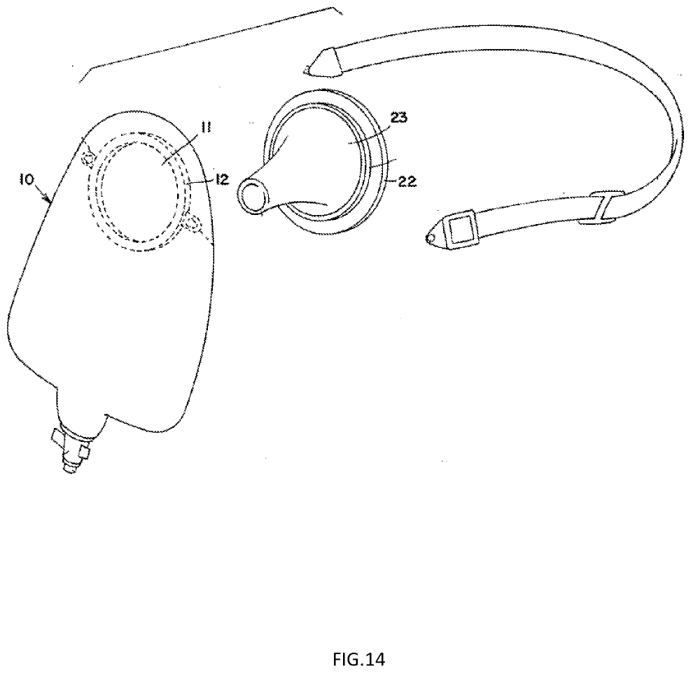

FIG. 15 shows a schematic view of another prior-art device.

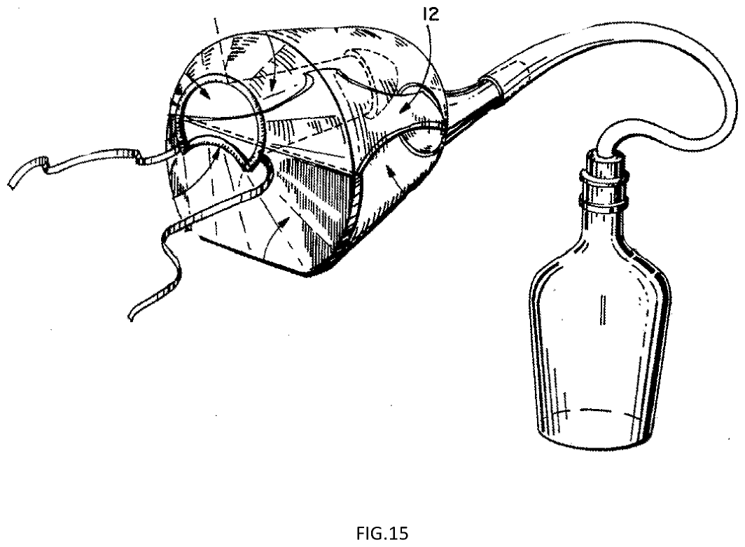

The foregoing and other features and advantages of illustrated embodiments of the present invention will be more readily apparent from the following detailed description, which proceeds with reference to the accompanying drawings.

### DETAILED DESCRIPTION OF THE ILLUSTRATED EMBODIMENTS

The foregoing and other technical contents, features and advantages of the present invention will be illustrated in detail by way of exemplary embodiments in the following paragraphs with reference to the accompanying drawings.

The drawings accompanying with the specification show the structural features of elements for the present invention, which may be depicted in a size or proportion to be easily understood by those skilled in the art without changing the subject matter of the present invention.

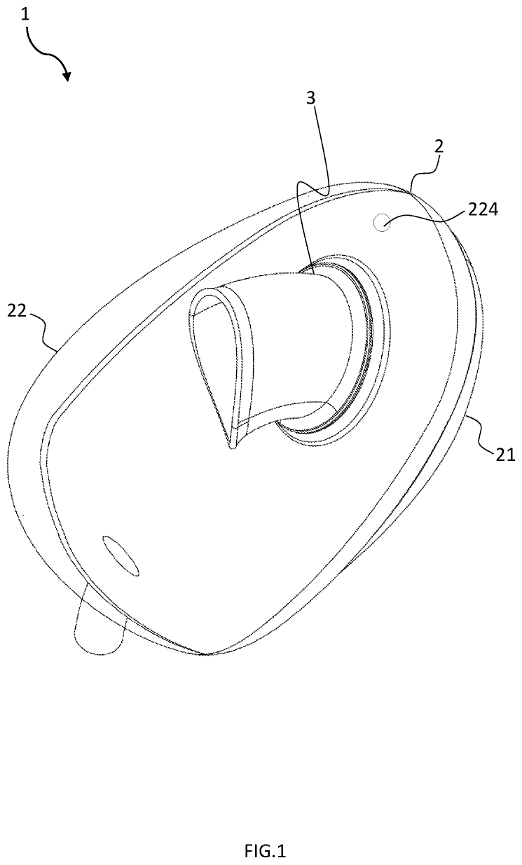

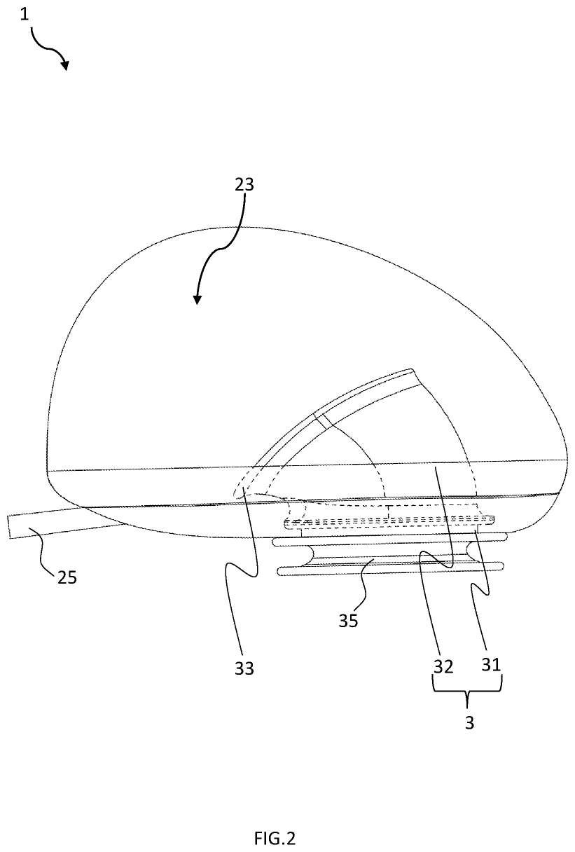

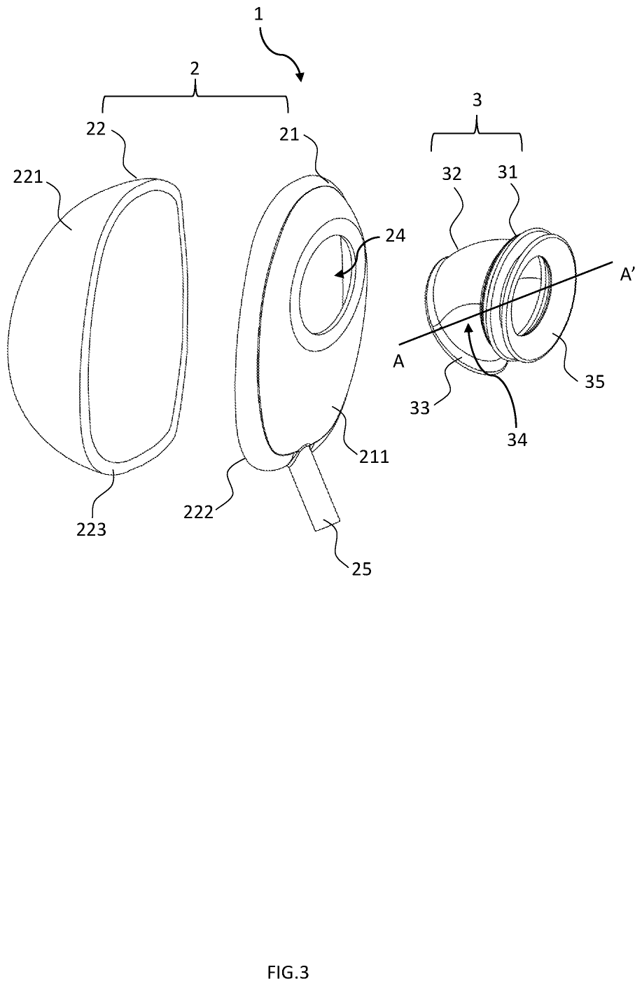

FIGS. 1 through 4 show a first embodiment of a urine collector 1 of the present invention, which can be used to collect the urine discharged from the genital organ (penis) of a male user. As shown, the urine collector 1 generally includes a container 2 and a short guide tube 3.

According to the first embodiment, the container 2 is constructed of a proximal shell 21 and a distal shell 22. The proximal shell 21 has a first wall 211 and a circumferential sealing portion 222 around the first wall 211. The distal shell 22 has a second wall 221 and a circumferential edge 223 around the second wall 221 and corresponding to the circumferential sealing portion 222. The circumferential sealing portion 222 can be placed in watertight engagement with the circumferential edge 223. The proximal shell 21 and the distal shell 22 can be assembled together through the engagement of the circumferential sealing portion 222 and the circumferential edge 223, thereby forming the container 2, which defines a temporary storage space 23 between the proximal shell 21 and the distal shell 22. In use, the first wall 211 faces towards the lower abdomen or crotch of a male user, close to the male genital organ. To avoid skin allergies, the proximal shell 21 is preferably made of a biocompatible material. The distal shell 22 is preferably made of a transparent, non-deformable material, so that the interior of the container 2 and the genital hygiene can be observed from the outside, and the container 2 is not easily squeezed, so that the volume of the temporary storage space 23 can be maintained. As such, the collector can be used in comfort, and skin allergies, eczema or ulceration can be prevented. The first wall 211 defines an opening 24 communicating with the temporary storage space 23 for receiving the short guide tube 3, and is provided with an outlet tube 25, through which the urine contained in the temporary storage space 23 can be discharged. A vent 224 is defined at the second wall 221 and located at a level higher than the outlet tube 25. This enables the urine contained the container 2 to be discharged more easily. In addition, the container 2 can be prevented from high humidity, and the odor released from the temporary storage space 23 can be reduced.

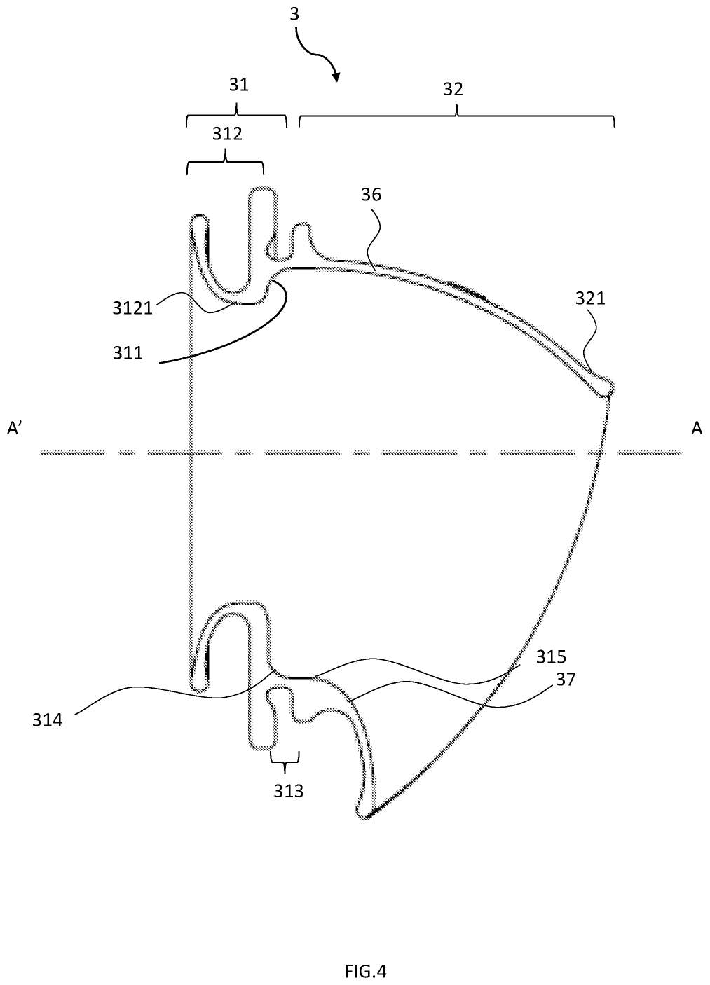

The short guide tube 3, which includes a connection section 31 and a main section 32, can be assembled into the container 2. For ease of illustration, the short guide tube can be divided into an upper part 36 and a lower part 37 (see FIG. 4). The connection section 31 can be engaged with a portion of the proximal shell 21 around the opening 24 to ensure a watertight connection between the container 2 and the short guide tube 3, so that leakage of urine and vapor thereof contained in the container 2, which is likely to wet skin or clothing to cause discomfort, can be prevented.

The connection section 31, located at one end of the short guide tube 3 close to the lower abdomen of a male user, has a neck portion 312 and a grooved portion 313. The short guide tube 3 has an inner surface 311, to which a tangent thereof changes as being made along the inner surface, such that an annular surface area 1321 with a minimum diameter is formed at the neck portion 312. The space defined by the annular surface area 1321 allows the genital organ (penis) of a male user to insert therethrough easily, resulting in a loose fit between the short guide tube 3 and the male genital organ, so that allergic inflammation and ulceration due to a long-term tight fit can be avoided. Furthermore, an inflexion surface area 314, where the rate of change of gradient (slope) of a tangent thereof is substantially zero, extends from the annular surface area 1321 and is formed at the grooved portion 313. The inflexion surface area 314 following the annular surface area 1321 causes a first difference of height on the inner surface of the short guide tube 3. Due to the gravity, the height difference can hinder the urine contained in the container 2 from flowing back.

As shown, the grooved portion 313 is asymmetrical about a central axis thereof, wherein the lower part of the grooved portion 313 has the inflexion surface area 314, which is followed by a flow directing surface area 315, to which a tangent thereof is negative in gradient (i.e. the tangent is going downwards in the direction of the distal end of the short guide tube). The negative gradient of the flow directing surface area 315 makes a distinctive downward bend, causing a second difference of height on the inner surface of the short guide tube 3. Even though the user is lying on a bed, since the outlet tube 25 is located between two legs of the user, the lower part 37 of the short guide tube 3 is still higher than the lower end of the outlet tube 25 (the first wall 211 extends at an angle of about 30 degrees to the horizontal line), the urine cannot flow back from the outlet tube 25 to the short guide tube 3. When the user is sitting or standing, there may be a little urine retained in the inflexion surface area 314. Upon urine continue to be discharged from the user, the urine contained in the inflexion surface area 314 can be forced to flow past the flow directing surface area 315. For this reason, the present invention does not need a tight fit structure as commonly used in conventional devices, which is liable to cause damages to the genital skin.

The main section 32 extends from the connection section 31 and can be located in the temporary storage space 23, wherein the main section 32 is formed with a top downwardly curved edge 321 and a bottom downwardly curved edge 33 that has a radius of curvature less than the top downwardly curved edge 321. The bottom downwardly curved edge 33 is at a level higher than the outlet tube 25 regardless of the postural condition of a user: standing, sitting or lying. Therefore, the urine discharged from a male user has a tendency to flow into the temporary storage space 23 via the bottom downwardly curved edge 33 and is unable to flow back over the main section 32, more specifically the bottom downwardly curved edge 33, to enter the short guide tube 3.

To use the collector more comfortable, the short guide tube 3 can be made of a flexible material, such as silicone, and the main section 32 can be configured with a curved surface 34 extending towards the outlet tube 25 and conforming to a male genital organ in natural state. For the bottom downwardly curved edge 33, which is curved towards the first wall 211, it can prevent over friction between a male genital organ and the distal end of the short guide tube 3. To prevent the testicles of a male user from being compressed, a buffer section 35 extending in a direction opposite to the main section 32 can be provided, which can be brought in elastic contact with the lower abdomen or crotch of the male user, and thus there exists a space to accommodate the testicles.

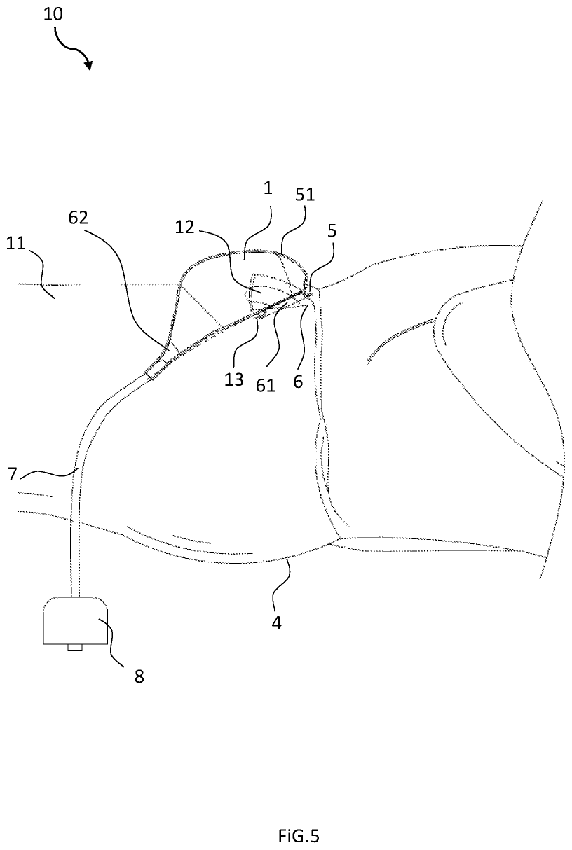

In this embodiment, the proximal shell 21 and the distal shell 22 are detachably assembled to form the container 2, so that the interior of the container 2 can be cleaned thoroughly to prevent breeding of germs and release of odor. As such, the container 2 can be used many times, thus reducing cost and meeting the trend of environmental protection. In use, the collector 1 can be located on the surface of the lower abdomen or crotch of a user to be aligned at an angle of about 30 degrees to the trunk of the user's body. Due to the gravity, the urine contained in the container 2 can be prevented from flowing back to the short guide tube 3 irrespective of the user's postural condition: standing, sitting or lying. FIG. 5 shows a urine kit 10 including a collector 1, a piece of underwear 4, a hose 7, and a storage bag 8, wherein the male user 11 is a bedridden patient. To facilitate medical staff taking care of patients, and to avoid disadvantages of prior-art devices, the hose 7 and the storage bag 8 can be fixed near the bed. The urine kit 10 can be operated easily and safely. The risk resulting from a catheter being fitted into the urethra of a male user as in the existing technology can be avoided.

The underwear 4 includes a main body 5 and an auxiliary part 6. The main body 5 has a crotch section 51 corresponding to the lower abdomen or crotch of the male user 11. The auxiliary part 6 is disposed at the inside of the main body 5 to define a space between the main body 5 and the auxiliary part 6. The collector 1 can be located in the space via an opening thereabove. The auxiliary part 6 defines a first hole 61 and a second hole 62. The first hole 61 allows the short guide tube 3 to pass through the auxiliary part 6 to be assembled into the collector 1, whereas the short guide tube 3 allows the genital organ 12 of the male user 11 to enter the collector 1. The second hole 62, which has a dimension greater than the outlet tube 25, is defined at the auxiliary part 6 and/or the underwear 4 to allow the outlet 25 to extend out of the underwear 4 to be connected with a hose 7, so that the urine contained in the collector 1 can be discharged into the storage bag 8. The urine discharged from the male user can enter the temporary storage space 23 via the bottom downwardly curved edge 33, and then flow into the storage bag 8 by way of the hose 7. In addition, the bottom downwardly curved edge 33 of the short guide tube 3, which is higher than the outlet tube 25, can prevent the urine contained in the temporary storage space 23 from entering the short guide tube 3.

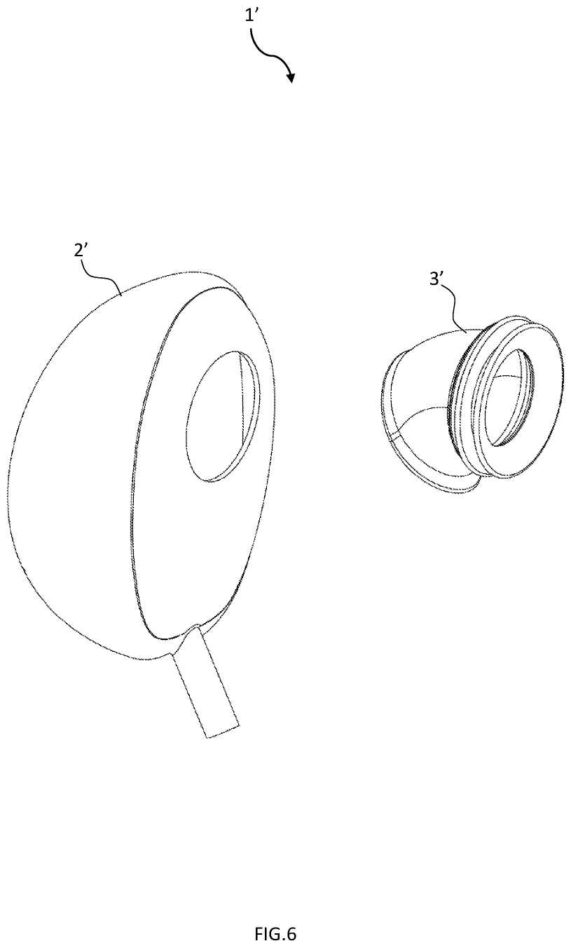

FIG. 6 shows a second embodiment of the collector, wherein description for parts the same as the first embodiment is omitted. The second embodiment is different from the first embodiment in that the container 2′ of this embodiment is an inseparable, integral body and formed of a polymeric material. The short guide tube 3′ is detachably assembled into an opening of the container 2′. Particularly, the inner surface of the container 2′ can be flipped out to be exposed to the outside for an easier cleaning, or the interior of the container 2′ can be cleaned through the opening. On the other hand, to facilitate observation of the internal state of the collector, the container 2′ can be made of a transparent, plastic material. Due to the simple structure, the manufacturing cost can be reduced.

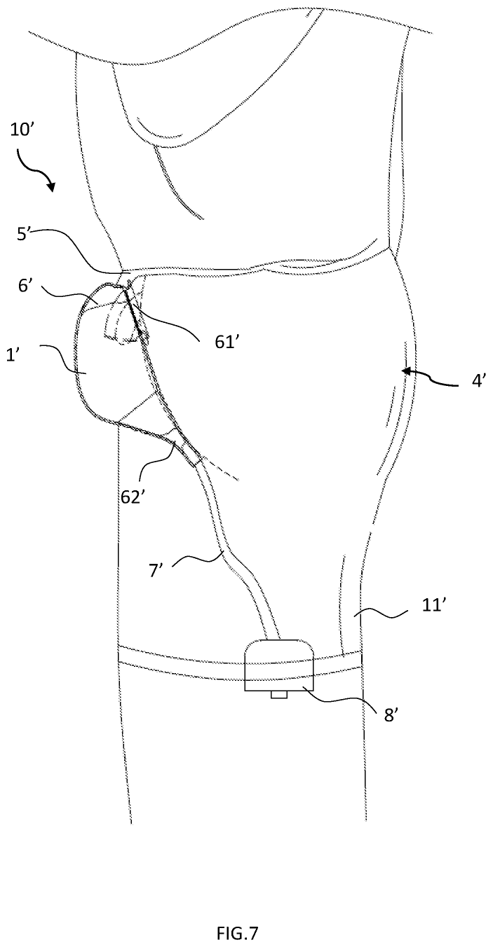

FIG. 7 shows a working view of a urine kit 10′. As shown, the kit 10′ includes a collector 1′ according to the second embodiment of present invention, a piece of underwear 4′, a hose 7′, and a storage bag 8′. A user 11′ using the kit 10′ can be a patient who is standing, taking a wheelchair, or driving a car. For ease of use, the storage baggage 8′ can be tied at a leg of the user. The storage bag 8′ can be concealed if the user wears loose trousers.

The underwear 4′ includes a main body 5′ and an auxiliary part 6′. The auxiliary part 6′ is disposed at the front side of the main body 5′ to define a space between the main body 5′ and the auxiliary part 6′. The collector 1′ can be located in the space via an opening communicating with the space. The main body 5′ defines a first hole 61′ for being fitted with a short guide tube 3′, while the auxiliary part 6′ defines a second hole 62′ to allow an outlet tube to extend therethrough. It is noticed that the opening, the first hole or the second hole can be defined at the main body of the underwear, the auxiliary body, or between the main body and the auxiliary body.

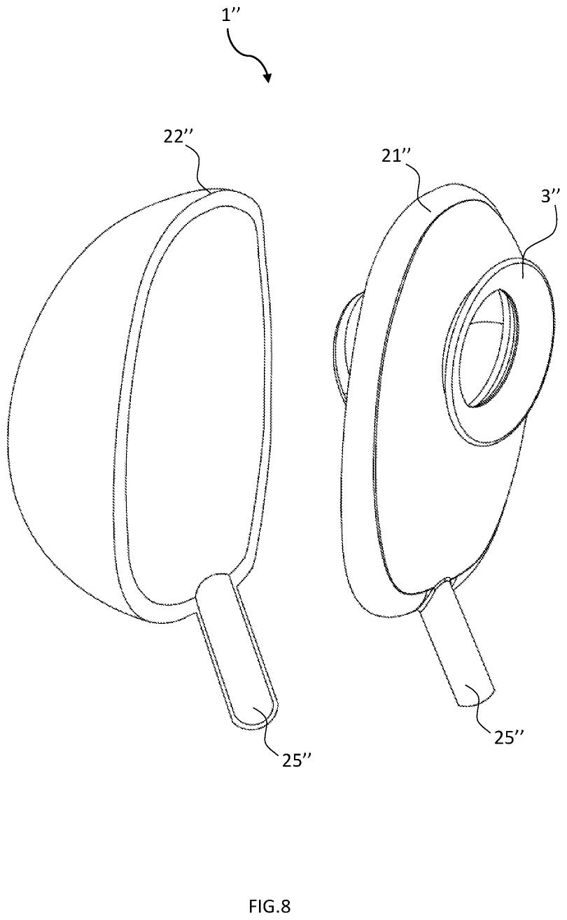

FIG. 8 shows a third embodiment of the collector of the present invention. The collector 1″ is different from the previous embodiments in that the proximal shell 21″ and the short guide tube 3″ are formed integrally, whereas the outlet tube is divided into two half parts 25″, one of which is at the proximal shell 21″ whereas the other of which is at the distal shell 22″. The two half parts 25″ can be matched to become a watertight tube. A hose can be fitted over the outlet tube to further prevent urine leakage.

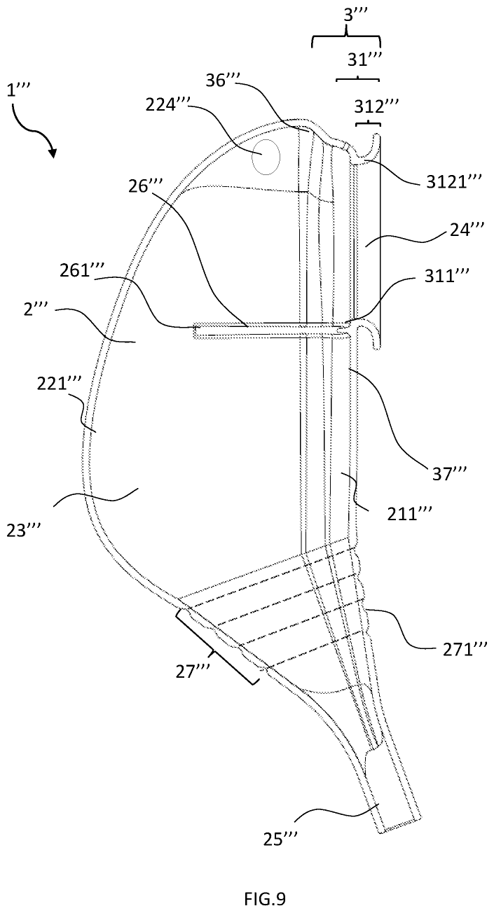

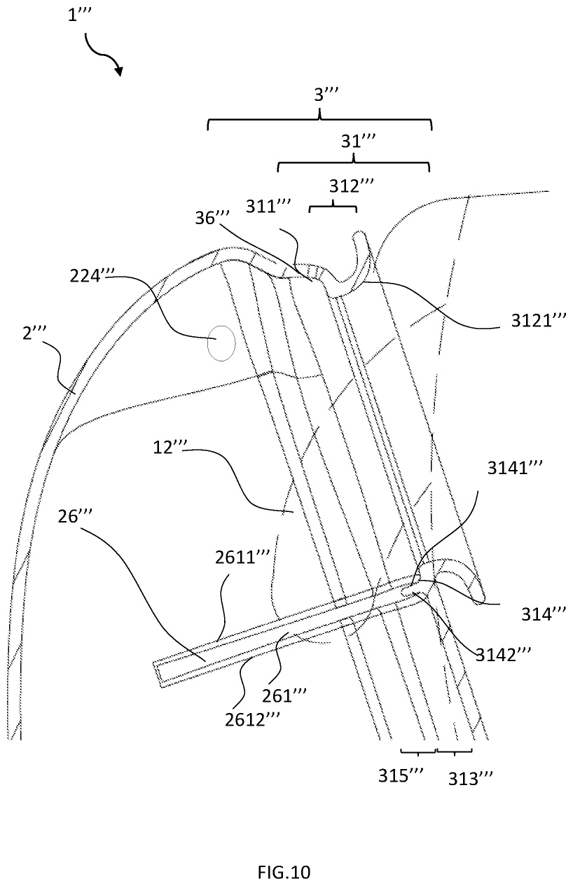

FIG. 9 shows a fourth embodiment of the collector of the present invention. FIG. 10 shows an enlarged fragmentary view of the collector shown in FIG. 9. This embodiment is different from the previous embodiments in that the first wall 211′″, the second wall 221′″, and the short guide tube 3′″ are formed integrally. The first wall 211′″ and the second wall 221′″ constitute the collector 2′″. The temporary storage space 23′″ is defined between the first wall 211′″ and the second wall 221′″. The first wall 211′″ defines an opening 24′″, through which the short guide tube 3′″ are communicated with the temporary storage space 23′″. The outlet tube 25′″, which communicates with the temporary storage space 23′″, is formed at a location distal from the opening 24′″ for discharging the urine contained in the temporary storage space 23′″. Particularly, a barrier 26′″, which extends from an inner surface of the first wall 211′″ close to the opening 24′″, is provided so that the urine at the outlet tube 25′″ can be blocked from flowing back to the opening 24′″.

In this embodiment, the barrier 26′″ may include two parallel segments 261′″, between which a gap is defined. Each segment 261′″ extends from a location corresponding to the opening 24′″ of the container in a direction opposite to the opening 24′″, decreasing in height, so as to prevent the urine from flowing back to the opening 24′″. The container 2′″ is formed with a bellows section 27′″, composed of multiple wavy folds 271, between the barrier 26′″ and the outlet tube 25′″, so as to increase the flexibility of the container 2′″, thus increasing the service life of the container 2′″.

For ease of illustration, the short guide tube portion 3′″ can be divided into an upper part 36′″ and a lower part 37′″. The short guide tube 3′″ has an inner surface 311′″, to which a tangent thereof changes in gradient as being made along the surface, wherein an annular surface area 3121′″ with a minimum diameter is formed. The space defined by the annular surface area 3121′″ allows the genital organ of a male user to insert therethrough easily, resulting in a loose fit between the short guide tube portion 3′″ and the male genital organ, so that occurrence of allergic inflammation and ulceration due to a long-term tight fit can be avoided.

Furthermore, an inflexion surface area 314′″, where the rate of change of gradient (slope) of a tangent thereof is substantially zero, extends from the annular surface area 3121′″. The inflexion surface area 314′″ following the annular surface area 3121′″ causes a first difference of height on the inner surface of the short guide tube portion 3′″. Due to the gravity, the height difference can hinder the urine contained in the container 2′″ from flowing back. A flow directing surface area 315′″, which extends from the inflexion surface area 314′″, is formed at the intersection of the short guide tube 3′″ and the barrier 26′″. Furthermore, a projection with an upper surface 3141′″ and a lower surface 3142′″ is formed at the inflexion surface area 314′″. In this embodiment, each segment 261′″ of the barrier 26′″ is composed of a first layer section 2611′″ corresponding to the upper surface 3141′″ of the projection, and a second layer section 2612′″ parallel to the first layer section 2611′″ and corresponding to the lower surface 3142′″ of the projection. Of course, those skilled in the art may understand that a single segment or a surrounding segment along the inner surface of the container can be provided without hindering the implementation of the present invention.

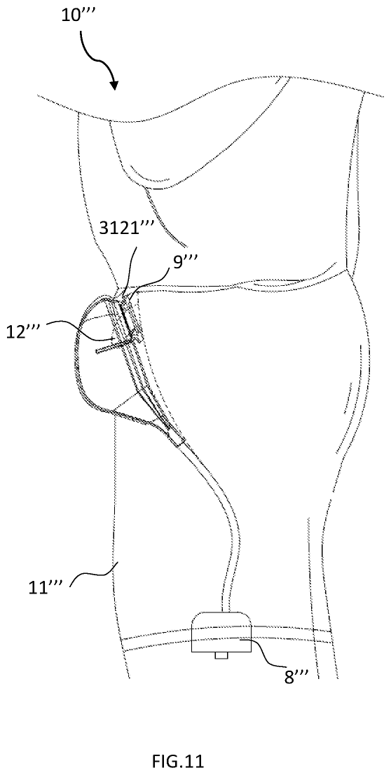

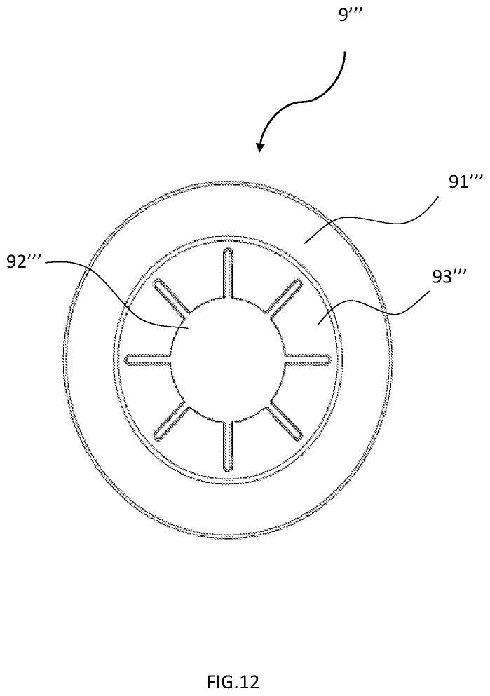

For a male user whose genital organ has been shrunk significantly, the urine kit 10′″ can be further provided with an adaptive member 9′″ (see FIGS. 11 and 12), which has a hydrophobic outer surface and can be fixed at the annular surface area 3121′″ of the short guide tube 3′″. The adaptive member 9′″ includes an engagement portion 91′″ that defines a central opening 92′″, and a plurality of flexible tabs 93′″ spaced evenly and extending from the engagement portion 91′″ towards the central opening 92′″. Thus, when the genital organ of a male user is inserted into the container, each flexible tab 93′″ can be forced to yield individually while being slightly elastic contact with the male genital organ. Due to the surface tension, the urine is difficult to flow back through the gaps between the flexible tabs 93′″, but can flow along the flexible tabs to drop into the container. The user 11′″ can be a patient who is standing, taking a wheelchair, or driving a car. For ease of use, the storage baggage 8′″ can be tied at a leg of the user. If the user wears loose trousers, the storage bag 8′″ can be concealed. This allows the user to be cared with dignity.

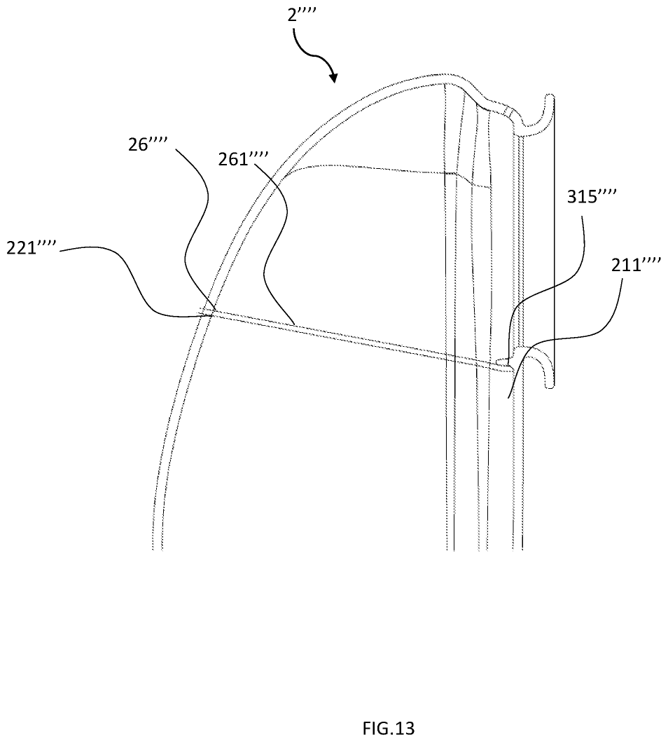

FIG. 13 shows a fifth embodiment of the collector, wherein description for parts the same as the previous embodiments is omitted. This embodiment is different from the fourth embodiment in that the barrier 26′″ includes at least one annular segment 261′″ extending along the inner surface of the first wall 211′″ and the inner surface of the second wall 221′″, wherein the segment 261′″ is not in a horizontal plane. As shown, the segment 261′″ together with the flow directing surface area 315′″ is located on a plane that slopes upwards in the direction of the second wall 221′″, so that the container 2′″ can have a larger space for receiving urine, thus increasing the blocking effect of the barrier 26′″ against urine backflow. In addition, the structure of the container can be simpler and easy to manufacture.

As a summary, the urine collector and kit of the present invention can be used to solve the inconvenience when persons or patients who are difficult to find or use a toilet when in hospital, driving a car, attending a meeting, etc. Furthermore, the urine collector includes a short guide tube configured with a bottom downwardly curved edge that is higher than the outlet tube of the collector to avoid the urine contained in the collector to flow out via the short guide tube, irrespective of whether the user is standing, lying or sitting. In addition to the bottom downwardly curved edge, the short guide tube is configured with a curved surface and a buffer section, which allow users to feel comfortable while using the collector, and leads the present invention to an ergonomic design. Other advantages of the present invention include: the collector can be cleaned easily and used many times to meet the trend of environmental protection; the urine collector and kit can be concealed easily to offer the patients comfort and dignity.

While the invention has been described with reference to the preferred embodiments above, it should be recognized that the preferred embodiments are given for the purpose of illustration only and are not intended to limit the scope of the present invention and that various modifications and changes, which will be apparent to those skilled in the relevant art, may be made without departing from the scope of the invention.

1. A urine kit for a male user, comprising:
a collector, including:
a container having a first wall and a second wall opposite to the first wall, and defining a temporary storage space between the first and second walls, the first wall facing a user's lower abdomen and defining an opening communicating with the temporary storage space, the container provided with an outlet tube communicating with the temporary storage space and extending from the first wall and/or the second wall; and
a short guide tube in watertight engagement with a portion of the first wall around the opening, the short guide tube including a connection section that has a neck portion and a grooved portion, the short guide tube having an inner surface to which the gradient of a tangent thereof changes as being made along the inner the surface, such that an annular surface area with a minimum diameter is formed at the neck portion; an inflexion surface area, where the rate of change of gradient of a tangent thereof is substantially zero, is formed at the grooved portion; a flow directing surface area, where the gradient of a tangent thereof is negative, extends from the inflexion surface area; and
a piece of underwear, including:
a main body having a crotch section; and
an auxiliary part disposed in the underwear to define a space between the crotch section of the underwear and the auxiliary part for accommodating the container and the short guide tube;
wherein a first hole is defined at the auxiliary part or the underwear to allow the short guide tube to pass therethrough, and a second hole having a dimension greater than the outlet tube is defined at the auxiliary part and/or the underwear.

2. The urine kit of claim 1, further comprising a hose and a storage bag, wherein the hose is adapted to be connected between the outlet tube and the storage bag.

3. The urine kit of claim 1, wherein the short guide tube further includes a main section extending from the connection section and located in the temporary storage space, the main section being formed with a top downwardly curved edge and a bottom downwardly curved edge that has a radius of curvature less than top downwardly curved edge, the bottom downwardly curved edge being at a level higher than the outlet tube, whereby the urine discharged from a male user flows into the temporary storage space via the bottom downwardly curved edge, whereas the urine contained in the temporary storage space is unable to flow back over the bottom downwardly curved edge to enter the short guide tube.

4. The urine kit of claim 1, wherein the short guide tube is formed integrally with the container.

5. The urine kit of claim 4, wherein at least one barrier is provided at an inner surface of the first wall of the container, close to the opening of the container, so that the urine contained in the temporary storage space is blocked from flowing back to the opening of the container, the barrier extending from the inflexion surface area of the short guide tube, the flow directing surface area being formed at the intersection of the short guide tube and the barrier.

6. A urine collector for a male user, comprising:
a container having a first wall and a second wall opposite to the first wall, and defining a temporary storage space between the first and second walls, the first wall facing a user's lower abdomen and defining an opening communicating with the temporary storage space, the container provided with an outlet tube communicating with the temporary storage space and extending from the first wall and/or the second wall; and
a short guide tube in watertight engagement with a portion of the first wall around the opening, the short guide tube including a connection section that has a neck portion and a grooved portion, the short guide tube having an inner surface, to which the gradient of a tangent thereof changes as being made along the inner the surface, such that an annular surface area with a minimum diameter is formed at the neck portion; an inflexion surface area, where the rate of change of gradient of a tangent thereof is substantially zero, is formed at the grooved portion; a flow directing surface area, where the gradient of a tangent thereof is negative, extends from the inflexion surface area.

7. The urine collector of claim 6, wherein the short guide tube further includes a main section extending from the connection section and located in the temporary storage space, the main section formed with a top downwardly curved edge and a bottom downwardly curved edge that has a radius of curvature less than the top downwardly curved edge, the bottom downwardly curved edge being at a level higher than the outlet tube, whereby the urine discharged from a male user flows into the temporary storage space via the bottom downwardly curved edge, whereas the urine contained in the temporary storage space is unable to flow back over the bottom downwardly curved edge to enter the short guide tube

8. The urine collector of claim 6, wherein the first wall of the container is integrally formed with the short guide tube.

9. The urine collector of claim 6, wherein the container is constructed of:
a distal shell having the second surface and a circumferential edge; and
a proximal shell having the first surface and a circumferential sealing portion capable of being in watertight engagement with the circumferential edge of the distal shell.

10. The urine collector of claim 6, wherein the short guide tube is formed integrally with the container.

11. The urine collector of claim 6, wherein the second surface defines at least one vent at a location higher than the outlet tube.

12. The urine collector of claim 6, wherein at least one barrier is provided at an inner surface of the first wall of the container, close to the opening of the container, so that the urine contained in the temporary storage space is blocked from flowing back to the opening of the container, the barrier extending from the inflexion surface area of the short guide tube, the flow directing surface area being formed at the intersection of the short guide tube and the barrier.

13. The urine collector of claim 12, wherein the barrier includes at least one segment extending from a location corresponding to the opening of the container, the segment decreasing in height as extending in a direction opposite to the opening.

14. The urine collector of claim 12, wherein the short tube is configured with a projection at the inflexion surface area, the projection having an upper surface and a lower surface, the segment of the barrier composed of a first layer section corresponding to the upper surface of the projection, and a second layer section parallel to the first layer section and corresponding to the lower surface of the projection.

15. The urine collector of claim 6, further comprising an adaptive member fixed at the annular surface area at the neck portion of the short guide tube, the adaptive member including an engagement portion that defines a central opening, and a plurality of flexible tabs spaced evenly and extending from the engagement portion towards the central opening.

16. The urine collector of claim 6, wherein the short guide tube further includes a buffer section extending from the connection section in a direction opposite to the main section, the buffer section capable of being elastic contact with a male user's lower abdomen such that a space is defined between the first wall and the user's lower abdomen so as to accommodate the male user's testicles.

17. The urine collector of claim 12, wherein the container is formed with a bellows section between the barrier and the outlet tube, so as to increase the flexibility of the container.

18. A short guide tube for use in a urine collector including a container that defines a temporary storage space therein and an opening communicating with the temporary storage space, and is provided with a an outlet tube communicating with the temporary storage space, the short guide tube capable of guiding the urine discharged from a male user to enter the temporary storage space, the short guide tube including:
a connection section in water tight engagement with a portion of the container around the opening, the connection section having:
a neck portion configured with an annular surface area with a minimum diameter at an inner surface of the short guide tube; and
a grooved portion, next to the neck portion, configured with an inflexion surface area extending from the annular surface area, and a flow directing surface area extending from the inflexion surface area, wherein the inflexion surface area is substantially zero in rate of change of gradient, and the flow directing area is negative in gradient; and
a main section extending from the connection section and located in the temporary storage space, the main section formed with a top downwardly curved edge and a bottom downwardly curved edge that has a radius of curvature less than the top downwardly curved edge to prevent the urine contained in the temporary storage space from entering the short guide.

19. The short guide tube of claim 18, wherein the short guide tube is made of a flexible material.

20. The short guide tube of claim 18, wherein the main section of the short guide tube is configured with a curved surface extending towards the outlet tube.
